(19) United States
(12) Patent Application Publication
(10) Pub. No.: US 2023/0211073 A1
(43) Pub. Date: Jul. 6, 2023

(54) INFUSION PUMP SECURITY SYSTEM

(71) Applicants: Baxter International Inc., Deerfield, IL (US); Baxter Healthcare SA, Glattpark (Opfikon) (CH)

(72) Inventors: Rajesh Swarnkar, Bangalore (IN); Gupta Ankur, Horamavu (IN); Soruban Manickalinga Thandapani, Whitefield (IN); David Fahmy Gorgi, Calumet City, IL (US); Marinoi Angela, Deerfield, IL (US); Saman Rais Alam Khan, Mumbai Central (IN); Prasad Deepak Digaskar, Kandivali East (IN); Paul Vincent O'Brien, Rochester, NY (US); Patil Sanskriti, Whitefiled main road (IN)

(21) Appl. No.: 18/092,611

(22) Filed: Jan. 3, 2023

Related U.S. Application Data
(60) Provisional application No. 63/329,145, filed on Apr. 8, 2022.
(60) Provisional application No. 63/295,704, filed on Dec. 31, 2021.

(51) Int. Cl. A61M 5/142

(52) U.S. Cl. CPC A61M 5/142; A61M 2005/16863

(57) ABSTRACT

A system and method for securing a pump are disclosed. The system includes a pump that contains a medicament delivery system, a processor operably coupled to the pump, a user interface in operable communication with the processor, a plurality of sensors in operable communication with the processor, and a housing. The housing includes a frame, a door, and a locking mechanism that can be selectively unlocked to allow the door to partially detach from the frame. The security system includes a swivel door assembly, which includes physical redundancies to selectively prevent access to the internal space of the swivel door assembly and a plurality of alarms. For example, in response to receiving an input from the plurality of sensors, the processor initiates at least one of the plurality of alarms to provide an indication to a user that an event has occurred.

### PRIORITY CLAIM

The present application claims priority to and benefit of U.S. Provisional Patent App. No. 63/295,704, filed Dec. 31, 2021, titled INFUSION PUMP SECURITY SYSTEM, and U.S. Provisional Patent App. No. 63/329,145, filed Apr. 8, 2022, titled INFUSION PUMP SECURITY SYSTEM, the entire contents of which are incorporated by reference herein and relied upon.

### BACKGROUND

Unauthorized tampering with medicament delivery systems, such as patient-controlled analgesia (“PCA”) pumps, to obtain pain medication, creates a risk to patient safety as well as inefficiencies in healthcare systems. Drug diversion by a malicious individual or clinician could result in a patient not receiving the necessary dose of medicament, which could be detrimental to the patient's outcome. Similarly, drug diversion can be costly and taxing on a health care system's finite resources. Current security systems trigger an alarm as soon as it detects potential unauthorized access to a pump. If an unauthorized user silences the alarm immediately, it would negate the auditory effect of the alarm. As a result, system administrators will not be aware of the alarm and the potential diversion. More specifically, the system administrators are unaware if an unauthorized user has potentially gained access to the drug secured by the pump and do not know what action has to be taken after an unauthorized user gains access to pump. As such, a security system that provides an indication of the unauthorized access and a system that remains in an alarm state until an authorized user receives the indication is desirable.

Moreover, in current security systems, when an unauthorized user is able to make contact with the medicament delivery system, using external tools, medicament can be diverted. Therefore, a security system that redundantly blocks the medicament delivery system from external tools is desirable. Additionally, a need exists for a system that provides an authorized user of the system an indication and details of unauthorized use.

### SUMMARY

Example systems, methods, and apparatus are disclosed herein for a system and method for securing a pump. The system includes a pump that contains a medicament delivery system, a processor integrated and operably coupled to the pump, a user interface in operable communication with the processor, a plurality of sensors in operable communication with the processor, and a housing. The housing includes a frame, a door, and a locking mechanism that can be selectively unlocked to allow the door to partially detach from the frame. The security system includes a swivel door assembly, where the swivel door assembly includes physical redundancies to selectively prevent access to the internal space of the swivel door assembly and a plurality of alarms. For example, in response to receiving an input from the plurality of sensors, the processor initiates at least one of the plurality of alarms to provide an indication to a user of the system that an event has occurred. The example systems, methods, and apparatus are configured to prevent unauthorized user access to pumps. Additionally, the example systems, methods, and apparatus are configured to provide an authorized user with indications and details of unauthorized use on a user interface screen. As such, the disclosed systems, methods, and apparatus increase efficient infusion administration by preventing medicament diversion.

In light of the disclosure herein, and without limiting the scope of the invention in any way, in a first aspect of the present disclosure, which may be combined with any other aspect listed herein unless specified otherwise, a patient-controlled analgesia pump includes an actuator, a syringe holder, a display screen, a security door assembly, a security frame assembly, a swivel door assembly, a plurality of sensors, including at least one occlusion sensor and at least one downstream inline pressure sensor, a syringe flange, a syringe push button, a syringe barrel clamp, a syringe pump lever, a processor and a memory storing instructions. The instructions, when executed by the processor, cause the processor to generate a syringe mis-loaded alarm when a syringe is mis-loaded into the PCA pump, generate a syringe load error alarm when there is a syringe load error in the PCA pump, generate a door unlocked alarm when the security door assembly is unlocked from the security frame assembly, generate a downstream occlusion alarm when there is a downstream occlusion in the PCA pump. Also, the alarm is an auditory, graphical, visual, and/or tactile notification, or a combination thereof, user-inputted authentication commands allow the user to silence and/or stop the alarm or alarms.

In a second aspect of the present disclosure, which may be combined with any other aspect listed herein unless specified otherwise, the syringe mis-loaded alarm is triggered when the security door assembly is closed and confirmed locked by the user, the pump is in a delivery mode, and any of the following conditions are true, except when the syringe mis-loaded alarm is active: the syringe flange is removed; the syringe push button is not in contact with the occlusion sensor; the syringe barrel clamp is opened; the syringe pump lever is activated; and/or the syringe push button detects high pressure reading.

In a third aspect of the present disclosure, which may be combined with any other aspect listed herein unless specified otherwise, the syringe mis-loaded alarm is triggered and the pump stops the delivery mode, when the pump is in delivery mode and any of the following conditions are true: the syringe flange is removed; the syringe push button is not in contact with the occlusion sensor; the syringe barrel clamp is opened; and/or syringe pump lever is activated.

In a fourth aspect of the present disclosure, which may be combined with any other aspect listed herein unless specified otherwise, the syringe load error alarm is triggered when the security door assembly is closed and confirmed locked by the user, the pump is not in a delivery mode, or is in a stopped state, the syringe push button detects a high pressure reading, and a syringe load point gets actuated.

In a fifth aspect of the present disclosure, which may be combined with any other aspect listed herein unless specified otherwise, the door unlocked alarm is triggered when the security door assembly is opened and the pump is in delivery mode.

In a sixth aspect of the present disclosure, which may be combined with any other aspect listed herein unless specified otherwise, the downstream occlusion alarm is triggered when the downstream in-line sensor detects pressure outside a user-inputted pressure range at about 1.0 meter downstream.

In a seventh aspect of the present disclosure, which may be combined with any other aspect listed herein unless specified otherwise, the security door assembly further comprises mating portions and the security frame assembly further comprises mating portions.

In an eight aspect of the present disclosure, which may be combined with any other aspect listed herein unless specified otherwise, the mating portions on the security door assembly selectively engage the mating portions on the security frame assembly to selectively couple the security door assembly and the security frame assembly.

In a ninth aspect of the present disclosure, which may be combined with any other aspect listed herein unless specified otherwise, the security door assembly further comprises ribs, grooves and/or contours on the internal surface of the security door assembly.

In a tenth aspect of the present disclosure, which may be combined with any other aspect listed herein unless specified otherwise, the security door assembly further comprises a receiver and the security frame assembly further comprises a lock assembly.

In an eleventh aspect of the present disclosure, which may be combined with any other aspect listed herein unless specified otherwise, the lock assembly selectively mates with the receiver.

In a twelfth aspect of the present disclosure, which may be combined with any other aspect listed herein unless specified otherwise, a patient-controlled analgesia pump includes an actuator, a syringe holder, a display screen, a security door assembly, including a receiver, a security frame assembly, including a lock assembly, a swivel door assembly, including a syringe pump lever in an upright and depressed orientation, and a plurality of sensors, including at least one occlusion sensor and at least one downstream inline pressure sensor. The security door assembly interlocks with the security frame assembly and the lock assembly mates with the receiver.

In a thirteenth aspect of the present disclosure, which may be combined with any other aspect listed herein unless specified otherwise, the swivel door assembly further includes a main assembly body to which the lever door pivotably couples, a swivel door that is pivotably attached to the assembly body and that includes side walls, a side wall plate coupled to the main assembly body so as to fully cover the syringe pump lever, a lever stopper, and a pull knob stopper.

In a fourteenth aspect of the present disclosure, which may be combined with any other aspect listed herein unless specified otherwise, the swivel door has an internal curvature that interlocks with the plunge lever and redirects force applied on the syringe pump lever, such that the plunge lever cannot be depressed when the swivel door is in a closed configuration relative to the main assembly body.

In a fifteenth aspect of the present disclosure, which may be combined with any other aspect listed herein unless specified otherwise, the lever stopper is pivotably attached to the main assembly body and interlocks with the syringe pump lever to retain it in an upright orientation. Also, the pull knob stopper includes a locking pin that mates with the lever stopper at a locking receptacle to selectively lock it when it interlocks with the syringe pump lever.

In a sixteenth aspect of the present disclosure, which may be combined with any other aspect listed herein unless specified otherwise, the pull knob lever includes a mechanism to orient the locking pin to engage with the locking receptacle, and the user can selectively inactivate the mechanism, so as to selectively disengage the locking pin and locking receptacle.

In a seventeenth aspect of the present disclosure, when the security door assembly and security frame assembly interlock and are in a closed configuration, the syringe holder is inaccessible.

In a eighteenth aspect of the present disclosure, which may be combined with any other aspect listed herein unless specified otherwise, when the security door assembly and security frame assembly interlock and are in a closed configuration, the display screen is accessible.

In a nineteenth aspect of the present disclosure, which may be combined with any other aspect listed herein unless specified otherwise, when the security door assembly and the security frame assembly interlock and are in a closed configuration, the swivel door assembly is inaccessible.

In a twentieth aspect of the present disclosure, which may be combined with any other aspect listed herein unless specified otherwise, when the security door assembly and security frame assembly interlock and are in a closed configuration, the actuator is inaccessible

In a twenty-first aspect of the present disclosure, any of the structure, functionality, and alternatives disclosed in connection with any one or more of FIGS. 1 to 13 may be combined with any other structure, functionality, and alternatives disclosed in connection with any other one or more of FIGS. 1 to 13.

In light of the present disclosure and the above aspects, it is therefore an advantage of the present disclosure to prevent physical access to pump components and provide an authorized user of the system an indication and details of unauthorized use.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

Some example apparatus embodiments of the invention, and example procedures for making and using one or more example embodiments, are described in detail herein and by way of example, with reference to the accompanying drawings (which are not necessarily drawn to scale with regard to any internal or external structures shown) and in which like reference characters designate like elements throughout the several views, and in which:

FIG. 1A illustrates a perspective view of a security door assembly, according to an example of the present disclosure.

FIG. 1B illustrates a detailed view of a striker assembly of the security door assembly, according to an example of the present disclosure.

FIG. 2 illustrates a perspective view of a security frame assembly, according to an example of the present disclosure.

FIG. 3A illustrates a perspective view of an infusion pump utilizing the security system, in the unlocked position, according to an example of the present disclosure.

FIG. 3B illustrates a perspective view of an infusion pump utilizing the security system, with the security door in the locked position, according to an example of the present disclosure.

FIGS. 4A and 4B illustrate an exploded perspective view of a swivel door assembly, according to an example of the present disclosure.

FIGS. 5A and 5B illustrate exploded perspective views of the interior of the swivel door assembly, according to an example of the present disclosure.

FIGS. 6A, 6B and 6C illustrate perspective views of the plunger stopper, according to an example of the present disclosure.

FIGS. 7A, 7B and 7C illustrate perspective views of an alternative plunger stopper, according to an example of the present disclosure.

FIGS. 8A, 8B and 8C illustrate perspective views of the pull knob stopper, according to an example of the present disclosure.

FIG. 9 illustrates a detailed view of a plunger stopper in a locked configuration with a pull knob stopper, according to an example of the present disclosure.

FIG. 10 illustrates a flow diagram of a syringe mis-loaded alarm sequence of the infusion pump security system, according to an example of the present disclosure.

FIG. 11 illustrates a flow diagram of a syringe load error alarm sequence of the infusion pump security system, according to an example of the present disclosure.

FIG. 12 illustrates a flow diagram of a door unlocked alarm sequence of the infusion pump security system, according to an example of the present disclosure.

FIG. 13 illustrates a flow diagram of a downstream occlusion alarm sequence of the infusion pump security system, according to an example of the present disclosure.

### DETAILED DESCRIPTION OF EXAMPLE EMBODIMENTS

The present disclosure generally describes a pump security system and methods of using the same that guard against unauthorized access to pump components. Physical components such as locks, enclosure assemblies, and/or sensors may be used to physically prevent unauthorized access to pump components. Additionally, virtual security measures such as alarms and passcodes may also be used to restrict access to pump components. Both the physical and virtual security components may be used in a complimentary fashion. In an example, sensors may detect tampering with medicament delivery, which may trigger an alarm. In the event of unauthorized tampering, a passcode is required to silence the alarms and alarms can be silenced only by the authorized users. This will alert a user that a tampering event is actively occurring or has taken place. The unauthorized user will be deterred hearing this alarm. In an additional example, plunger stoppers are disclosed that introduce physical redundancies that require the user to perform dual, simultaneous actions to divert the drug, which makes diverting the drug more difficult to do, thereby, increasing the security of the pump.

Additionally, alarm sequences that are triggered in response to various unauthorized events or pump abnormalities are disclosed. These alarms may aid in preventing tampering events and may provide tampering event solutions that instruct the user of the system the steps to perform when a tampering event takes place.

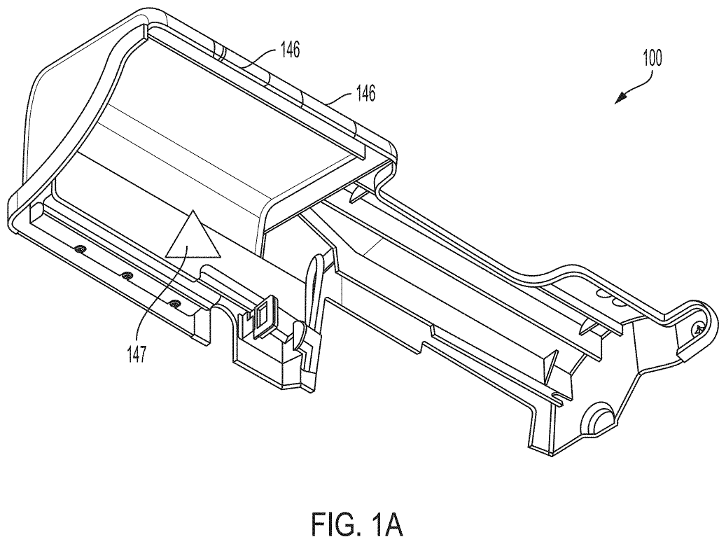

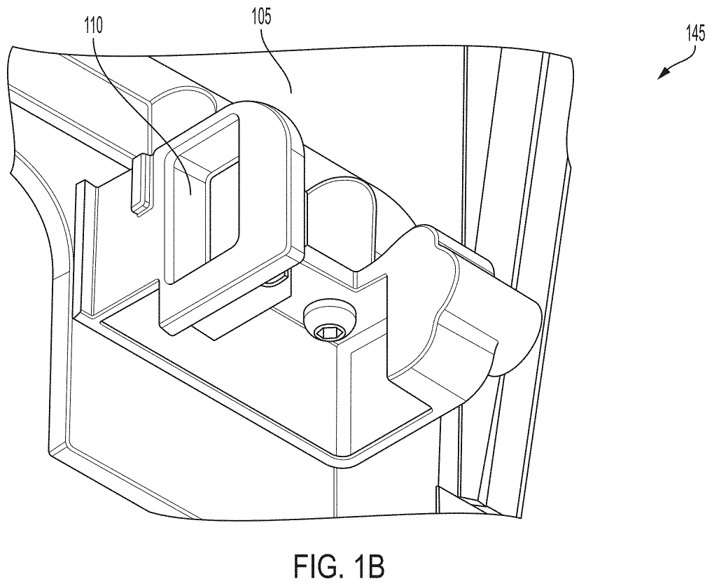

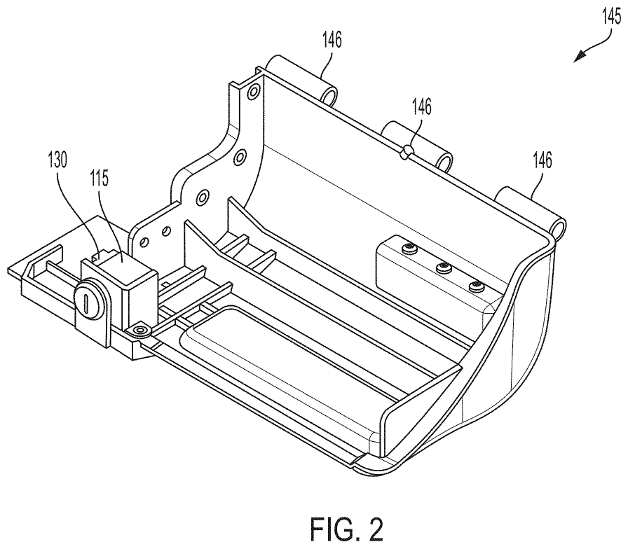

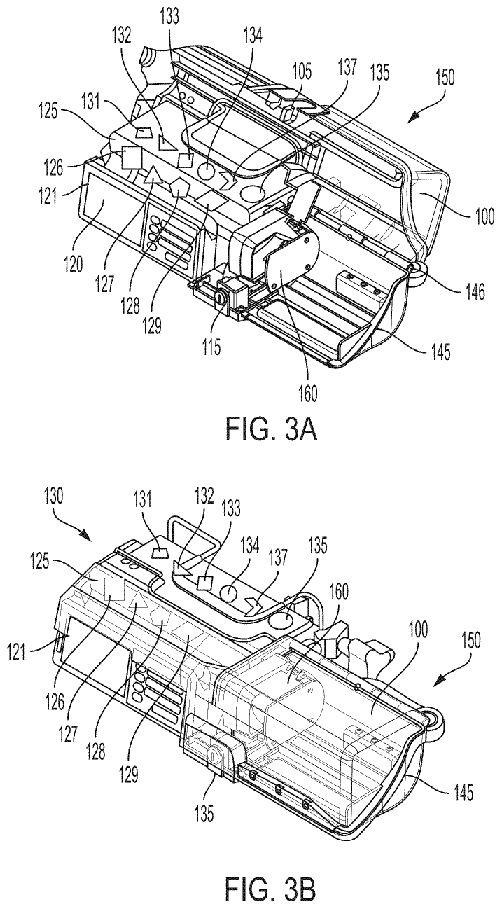

FIG. 1A illustrates a perspective view of the security door assembly 100, according to an example of the present disclosure. In an embodiment, the security door assembly 100 protects the syringe 132 (see FIG. 3A), barrel clamp 137 (see FIG. 3A) and plunger push button 135 (see FIG. 3A), thereby ensuring the drug is not accessible to the unauthorized person while the door is locked. The security door assembly may include a variety of ribs, grooves and/or contours 147 on the inside, and mating portions 146 on one of its edges. The security door assembly 100 may be a component of the pump 125 (see FIG. 3A). In an embodiment, the security door assembly 100 may include a plurality of sensors 131 (see FIG. 3A) in operable communication with a processor 126 (see FIG. 3A) to communicate various inputs to the processor 126. In an embodiment, the security door assembly may be arranged with a security frame assembly 145 (see FIG. 2) to create an enclosure for the pump 125 (see FIG. 3A). In an embodiment, the security door assembly 100 may enclose the entire pump thereby protecting the pump 125, display screen 121, and user interface 120 (see FIG. 3A).

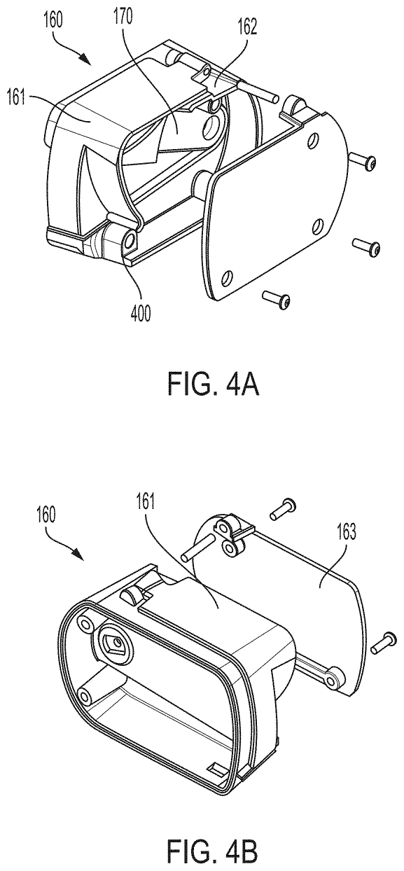

FIG. 1B illustrates a detailed view of a striker assembly 105 of the security door assembly 100, according to an example the present disclosure. In an embodiment, the security door assembly 100 may include a striker assembly 105 which can be reversibly locked when used in conjunction with a locking deadbolt assembly 115 (see FIG. 2). Specifically, a receiver 110 of the striker assembly 105 has a mating relationship with a deadbolt 130 (see FIG. 2) of the lock assembly 115 (see FIG. 2). The striker/dead bolt systems 105, 115 ensures the security door assembly 100 is retained in the locked position unless opened with a proper key. As a result, an unauthorized user cannot access the plunger push button 135 (see FIG. 3A) and plunger lever 170 (see FIG. 4A) for the purpose of unauthorized tampering such as drug diversion. In an embodiment, the lock assembly 115 has been selected to be of a strong and robust material so as to minimize the possibility of an unauthorized user being able to break the lock deadbolt 115 open.

FIG. 2 illustrates a perspective view of a security frame assembly 145, according to an example of the present disclosure. In an embodiment, the security frame assembly 145 ensures the security door sub-assembly is locked during infusion. The security frame assembly may include a lock dead bolt assembly 115 that is able to be operably arranged with the striker 105 of the security door assembly 100 to ensure the security door 100 is retained in a locked position unless opened with the proper key. Specifically, the lock assembly 115 includes a deadbolt 130, which mates with the receiver 110 on the striker 105. As such, in an embodiment, the security frame assembly 145 may be arranged with the security door assembly 100 to create an enclosure immediately adjacent the pump 125 (see FIG. 3A). To achieve this, in an embodiment, the security door assembly 100 and the security frame assembly 145 both include mating portions 146, which interlock so as to selectively couple the security door assembly 100 and the security frame assembly 145 while still allowing the security door assembly 100 and the security frame assembly 145 to pivot about one another around the mating portions 146.

FIG. 3A illustrates a perspective view of a pump 125 utilizing the security system 150, in the unlocked position, according to an example of the present disclosure. The pump 125 includes a memory 127, a processor 126, an actuator 128, a syringe holder 129, a display screen 121, a security door assembly 100, a security frame assembly 145, a swivel door assembly 160, sensors 131, and a syringe 132 with a syringe flange 133, a syringe push button 134, a plunger push button 135, and a syringe barrel clamp 137. In an embodiment, when the security door assembly 100 is unlocked and partially disengaged from the security frame assembly 145, the pump user interface 120 and plunger 155 are accessible to the user of the system 150. The security door assembly 100 and the security frame assembly 145 are integrated with the pump 125 and fastened to the pump 125 using fasteners (not shown) inaccessible for tampering. Once the security door is closed and locked, the fasteners become inaccessible to the unauthorized user to prevent tampering with the door 100, to access the syringe 132, and, therefore, the drug (not shown). In an embodiment, the user interface 120 may be a graphical user interface and/or audio user interface. Also, in an embodiment, the user interface 120 receives user commands and then relays the commands to the processor 126, which may access the memory 127 to retrieve executable instructions that may be stored in the memory 127. The processor 126 may be capable of storing and executing machine readable instructions and coordinate input received from the plurality of sensors 131. In an embodiment, the plurality of sensors are placed about the pump 125 in select locations to detect various conditions of the pump 125 and/or security components. In response to the sensors 131 receiving specific inputs, the processor 126 may initiate various alarm sequences.

In an embodiment, the user interface 120 of the pump 125 may provide guidance for the user to check, verify and analyze tamper events. Additionally, various alarms hint the user to check for tampering on the pump (damages, breaks, etc.). The pump 125 may also be able to receive a preprogrammed security code via the user interface 120. Once a user enters the security code to access the pump 125, the alarm hints the user to check for tampering to determine if any damage or breakage has occurred. In an embodiment, the processor 126 of the pump 125 may provide the user with a post-tampering event solution, which may be an intuitive workflow the user follows to unlock the pump immediately after a tampering event. The intuitive workflow may be provided on the user interface 120, on the display screen 121, to aid the user in the proper steps to take when the tampering event takes place (i.e. resolving alarms).

In an additional embodiment, the pump 125 may include an event history log that may include details of a tampering event such as the amount of medicament diverted and, as a result, the user can make an informed decision on the next actions to take. For example, based on the event history log, the user may decide to continue with the therapy, replace the syringe 132, or focus additional attention to of the user if excess drug was diverted to the patient.

FIG. 3B illustrates a perspective view of the infusion pump 125 utilizing the security system 150, with the security door 100 in the locked position, according to an example of the present disclosure. In the locked position, the security door 100 pivots about the mating components 146. The user then engages the locking deadbolt assembly 115 such that the deadbolt 130 mates with the receiver 110. In an embodiment, when the security door assembly 100 is in the locked position, the plunger push button 135 and other selected regions of the pump 125 are inaccessible.

In an embodiment, the locking deadbolt assembly 115 of the security door assembly 100 and security frame assembly 145 ensures a physical key is needed to lock and unlock the security system 150 after loading a syringe 132 to deliver a medicament. Additionally, ribs, grooves and/or contours 147 (see FIG. 1A) may be provided on the interior surface of the security door 100 to minimize the possibility of an unauthorized user inserting some object while the door 100 is already locked. This results in a more robust security system 150, which discourages unauthorized users from accessing plunger push button 135 and plunger lever 170 (see FIG. 4A) for the purpose of drug diversion or other unauthorized tampering activities.

FIGS. 4A and 4B illustrate an exploded perspective view of a swivel door assembly 160, according to an example of the present disclosure. In an embodiment, the swivel door 161 is positioned to cover the plunger lever 170, which makes the plunger lever 170 inaccessible when the security door 100 is closed and locked. In an embodiment, the swivel door assembly 160 may include a swivel door 161, a hinge 162, a plunger lever 170, a side wall plate 163 and means for reversibly attaching 164 the side wall plate 163 to the swivel door assembly 160. The side walls 163 make it more difficult for any external tool to access the plunger lever 170. As a result, the plunger lever 170 can only be depressed when the door is open. In an embodiment, the components of the swivel door assembly 160 are easily disassembled to increase ease of cleaning and/or disinfection.

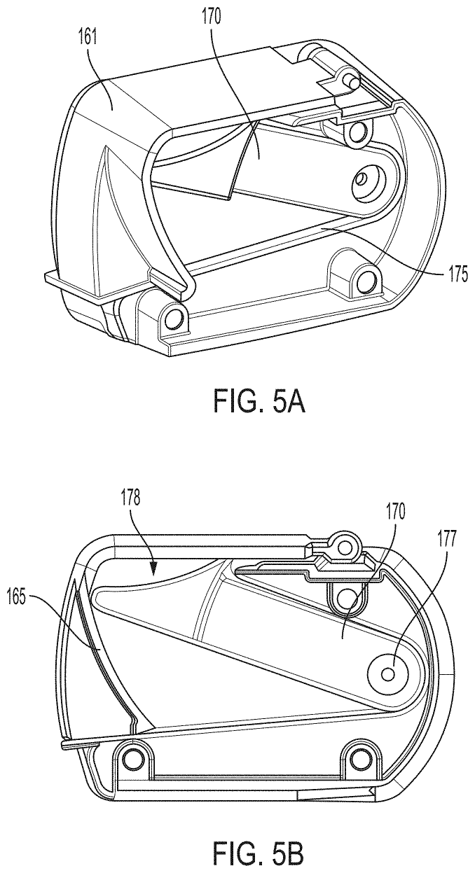

FIGS. 5A and 5B illustrate an exploded perspective view of the interior of the swivel door assembly 160, according to an example of the present disclosure. In an embodiment, the swivel door 161 rests on flat surface 175 and preferably has a curve 165 such that when force is applied on a lever 170 the force is transmitted to the flat surface 175 through the door 161 and closes the door 161. In an embodiment, the lever 170 is fixed at end one 177 to the swivel door assembly 160 and force is applied to end two 178, which is opposite end one 177 of the lever 170. Closing the swivel door 161 protects the plunger lever 170 from being depressed. As a result, the plunger lever 170 can be depressed only when the swivel door 161 is open.

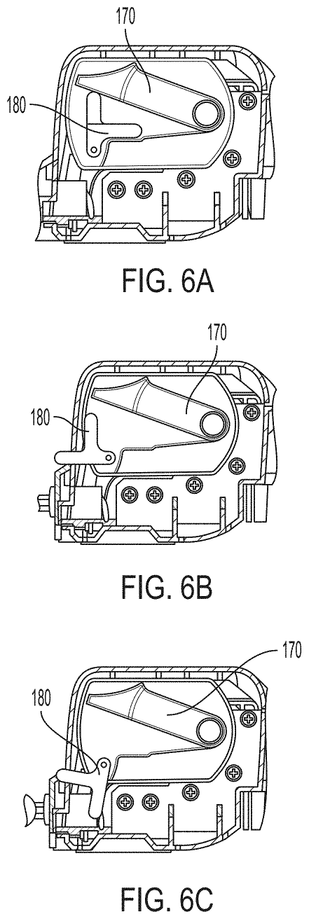

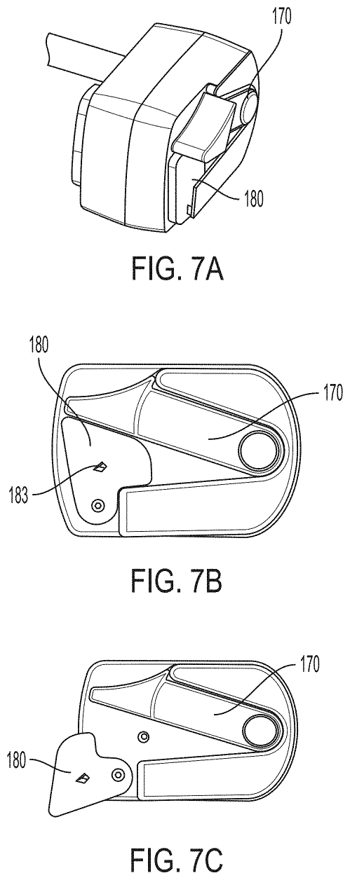

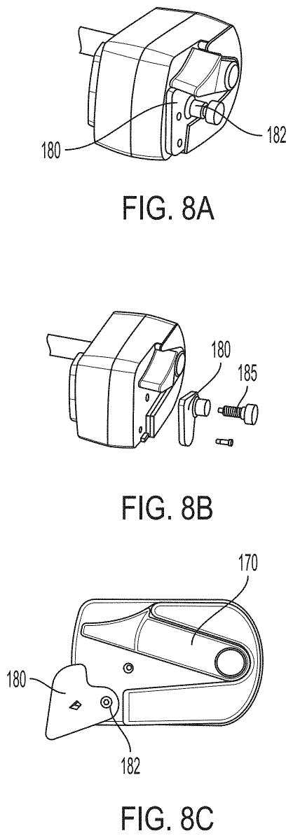

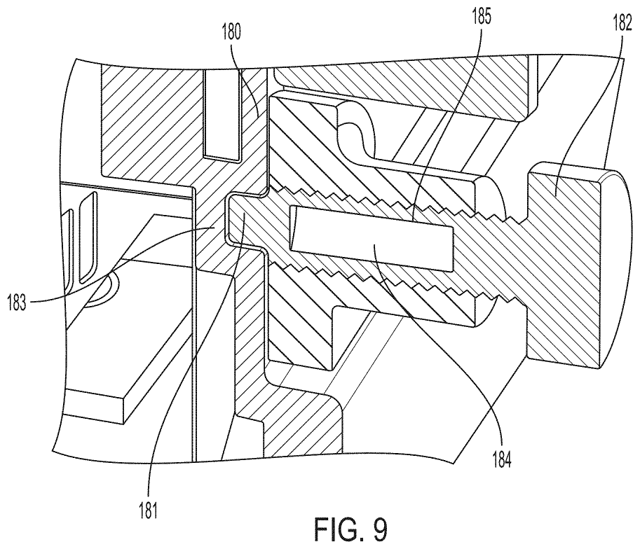

FIGS. 6A, 6B and 6C illustrate perspective views of the plunger stopper 180, according to an example of the present disclosure. In an embodiment, the stopper 180 prevents the movement of the plunger lever 170 by unauthorized users trying to access the plunger lever 170 without authorization. In an embodiment, if the security door 100 is closed and locked, even if the unauthorized user is able to access the plunger lever by an external tool, the user will not be able to depress the plunger lever 170 to divert the drug by pushing the plunger head (not shown). The plunger stopper 180 will prevent the depression of the plunger lever 170. Additionally, as seen in FIGS. 6B and 6C, the stopper 180 can only be rotated to its open position if the security door is unlocked and opened. As such, the plunger lever 170 is protected with the plunger stopper 180, which prevents the plunger lever 170 from being depressed while tampering is occurring. Specifically, the geometry of the plunger stopper 180 is one such that in certain configurations, the plunger stopper 180 interlocks with the plunger lever 170, such that the plunger lever 170 cannot be depressed. Additionally, the plunger stopper 180 includes a pull knob stopper 182 (see FIG. 8A). Also, ribs 181 (see FIG. 9) are provided on the surface of the pull knob stopper 182 to prevent the complete rotation of the plunger stopper 180. Specifically, the ribs 181 engage with mate ends 183 (see FIG. 7B) on the plunger stopper 180 such that the plunger stopper 180 cannot rotate. In an embodiment, the plunger head enclosure of the pump 125 may need to be modified to spatially accommodate the pull knob stopper 182.

FIGS. 7A, 7B and 7C illustrate perspective views of an alternative plunger stopper 180, according to an example of the present disclosure. In an embodiment, the stopper 180 prevents the movement of the plunger lever 170. The plunger lever 170 is protected with a rotatable plunger stopper 180, which prevents the plunger lever 170 from being depressed while tampering occurs. Additionally, the plunger stopper 180 includes a pull knob stopper 182 (see FIG. 8A). In addition, ribs 181 (see FIG. 9) are provided on the surface of the pull knob stopper 182 to prevent the complete rotation of the plunger stopper 180. Specifically, the ribs 181 engage with mate ends 183 on the plunger stopper 180 such that the plunger stopper 180 cannot rotate. In an embodiment, the plunger head enclosure of the pump 125 may need to be modified to spatially accommodate the pull knob stopper 182.

FIGS. 8A, 8B, and 8C illustrate perspective views of the pull knob stopper 182, according to an example of the present disclosure. In an embodiment, the pull knob stopper 182 prevents the movement of plunger lever 170. The pull knob stopper 182 requires the user to perform dual, simultaneous actions to depress the plunger lever 170 down and push the plunger head (not shown) to divert the drug as a security mechanism, creating physical redundancies. If the security door 100 is closed and locked, the pull knob stopper 182 makes it extremely difficult for the unauthorized user to depress the plunger lever 170 as the pull knob stopper 182 requires two operations to depress the plunger lever 170. In an embodiment, the pull knob stopper 182 requires the user to first pull the spring loaded knob 185 out (or knob with any similar mechanical design) and then with a second tool access and depress the plunger lever 170 down. The plunger lever 170 is further protected with a plunger stopper 180 which prevents the plunger lever 170 from being depressed while tampering is occurring. Additionally, the plunger stopper 180 includes a pull knob stopper 182. As previously noted, ribs 181 (see FIG. 9) are provided on the surface of the pull knob stopper 182 to prevent the complete rotation of the plunger stopper 180. Specifically, the ribs 181 engage with mate ends 183 on the plunger stopper 180 such that the plunger stopper 180 cannot rotate. In an embodiment, the plunger head enclosure of the pump 125 may need to be modified to spatially accommodate the pull knob stopper 182.

FIG. 9 illustrates a detailed view of a cross section of the plunger stopper 180 in a locked configuration with a pull knob stopper 182, according to an example of the present disclosure. As seen in FIG. 9, the pull ribs 181 on the pull knob stopper 182 to engage the mate ends 183 of the plunger stopper 180. The spring 184 in the spring loaded knob 185 keeps the pull ribs 181 engaged with the mate ends 183. In order to disengage the pull ribs 181 from the mate ends 183, the user must apply force opposite the direction of pull ribs 181 to overcome the tension created by the spring 184. As such, the plunger stopper 180 remains locked, and unable to rotate, until the user “pulls” the pull knob stopper 182, so as to disengage the mating relationship between the two stoppers 180, 182.

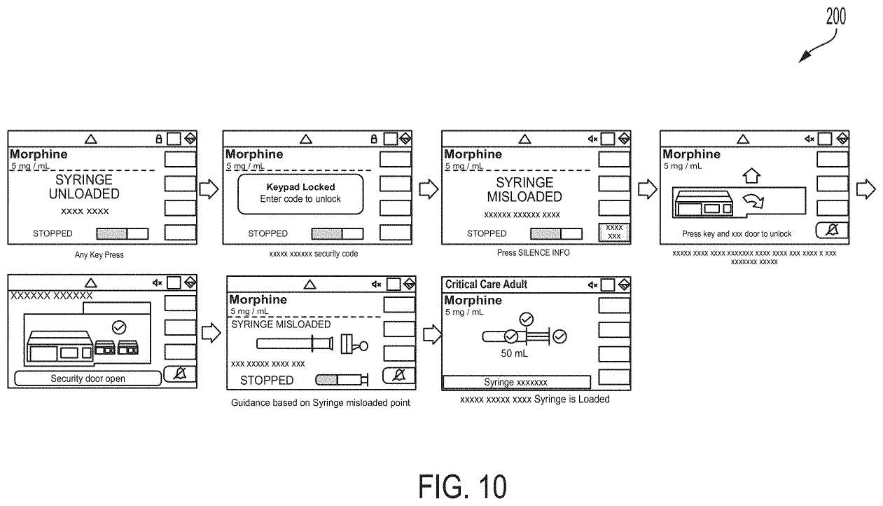

FIGS. 10 to 13 illustrate flow diagrams of alarm sequences of the infusion pump security system 150 in response to various inputs, received by a plurality of sensors 131, being received by the infusion pump security system 150, according to an example of the present disclosure. In an embodiment, providing an additional layer of authentication before attending to alarms can improve security of infusion pumps. The alarms, which may indicate possible tampering, can only be silenced and cleared once the authentic user enters a pre-programed passcode to access the pump. In an embodiment, tampering related alarms may be one of syringe load error, syringe mis-load, door unlocked, and downstream occlusion. These alarms will be generated as a result of drug diversion by an unauthorized user. However, these alarms may also be generated due to respective genuine reasons other than tampering by an unauthorized user. Once an authentic user enters the pre-programed passcode to access the pump, the alarm provides an indication to the user to check for tampering if any damage or breakage has been detected. The indication provided by the pump security system may be one of visual, auditory and/or tactile indication via the user interface 120 of the pump 125. In an embodiment, the user may program the alarm sequences to be synchronized or function in an overlapping manner (dual/multi-alarm approach). A dual/multi-alarm approach reduces the complexity of adding many alarms, which leads to alarm fatigue scenario in hospitals for the users.

FIG. 10 illustrates a flow diagram of a syringe mis-loaded alarm 200 sequence of the infusion pump security system 150, according to an example of the present disclosure. In an embodiment, the pump generates a high priority syringe load error alarm when the security door 100 is closed and confirmed to be locked by the user, the delivery mode is running, and any of the following conditions are true, except when the syringe mis-loaded alarm is active: syringe flange is removed; syringe push button not in contact with syringe pump occlusion sensor; syringe barrel clamp is opened; syringe pump lever is activated; and/or syringe push button detects high pressure reading.

In an additional embodiment, when the pump detects any of the following conditions while a delivery mode is running, the pump stops the delivery mode and generates a high priority syringe mis-loaded alarm: syringe flange is removed; syringe push button not in contact with syringe pump occlusion sensor; syringe barrel clamp is opened; and/or syringe pump lever is activated. Additionally, the user of the security system 150 may lock the security door 100 with a physical key after loading the syringe. Moreover, the security door 100 may have additional ribs, grooves, and/or contours 147 (see FIG. 1A) inside the security door 100 to minimize the possibility of an unauthorized user inserting an object into the housing while the door is already locked.

If an unauthorized user is still able to insert an object into the housing and actuate the syringe load points while the security door is locked with a loaded syringe, then as a further measure of security, additional alarms may be trigged.

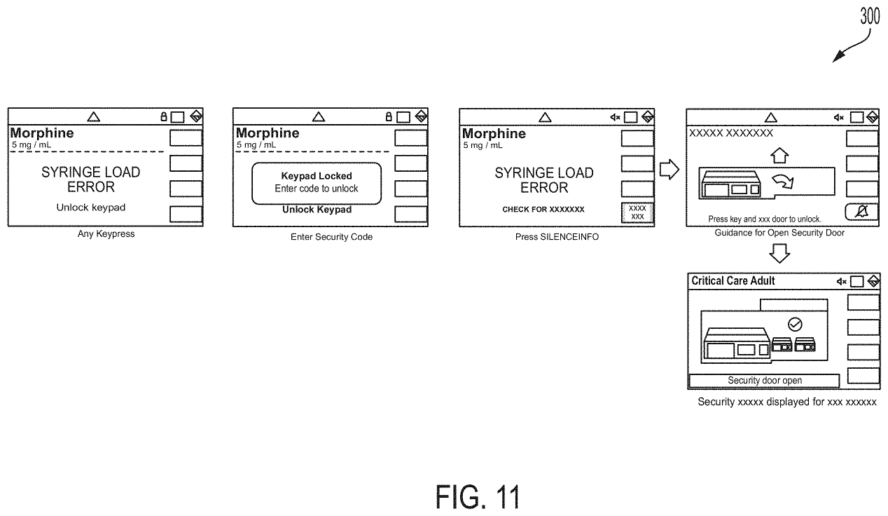

FIG. 11 illustrates a flow diagram of a syringe load error alarm sequence 300 of the infusion pump security system 150, according to an example of the present disclosure. Similar to the syringe mis-loaded alarm 200, the syringe load error alarm 300 is triggered upon an addition clause absent from the syringe mis-loaded alarm. In an embodiment, when the syringe push button detects a high pressure reading, the syringe load error alarm sequence 300 is triggered. If delivery mode is already running and the syringe push button detects high pressure reading, then a downstream occlusion alarm, instead of syringe mis-loaded alarm, is triggered.

Although similar, the difference between the syringe mis-loaded error alarm 200 and the syringe load error alarm 300 is that the syringe load error alarm 300 is generated when both of the following are true: delivery mode has not started or is in a stopped state, and any syringe load point gets actuated. Whereas the syringe mis-loaded alarm 200 is triggered when both of the following conditions occur: delivery mode is running (irrespective of whether fluid infusion is occurring) and any syringe load point gets actuated.

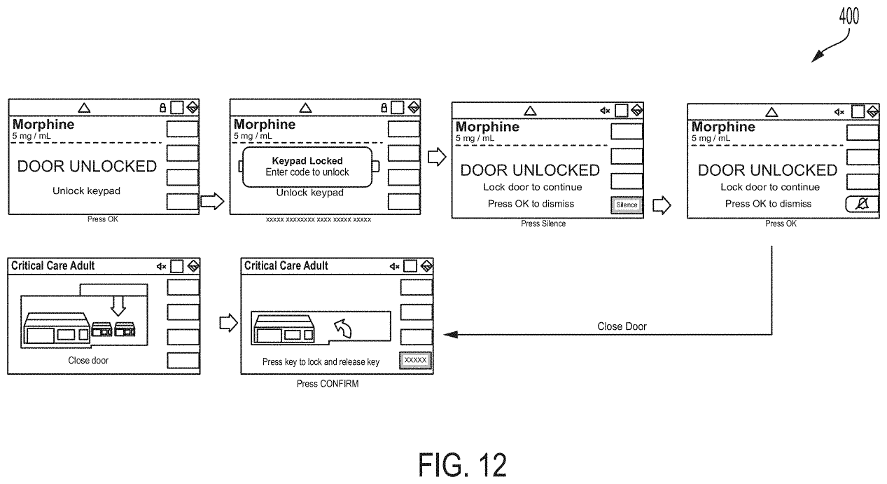

FIG. 12 illustrates a flow diagram of a door unlocked alarm 400 sequence of the infusion pump security system 150, according to an example of the present disclosure. In an embodiment, the pump stops the delivery mode and generates a high priority door unlocked alarm 400 when all of the following conditions occur: security door is opened and delivery mode is running. For an unauthorized user to generate the door unlocked alarm, either of the following need to occur: unauthorized user gets access to the physical key or unauthorized user breaks open the lock and opens the door. If an unauthorized user gains access to the physical key, LRS requirements are in place and the system user manual provides warnings for users and hospital personnel to keep the physical key in secured locations. If users or hospital personnel misplace the physical key and or an unauthorized user obtains the key, this scenario is outside the scope of the security systems ability to prevent unauthorized access. In an embodiment, the lock has been selected to be of a strong and robust material so as to minimize the possibility of an unauthorized user being able to break the lock open. The door unlocked alarm 400 is a further measure in case an unauthorized user is successful to open the door due to either unauthorized possession of a key or physical damage being done to the lock.

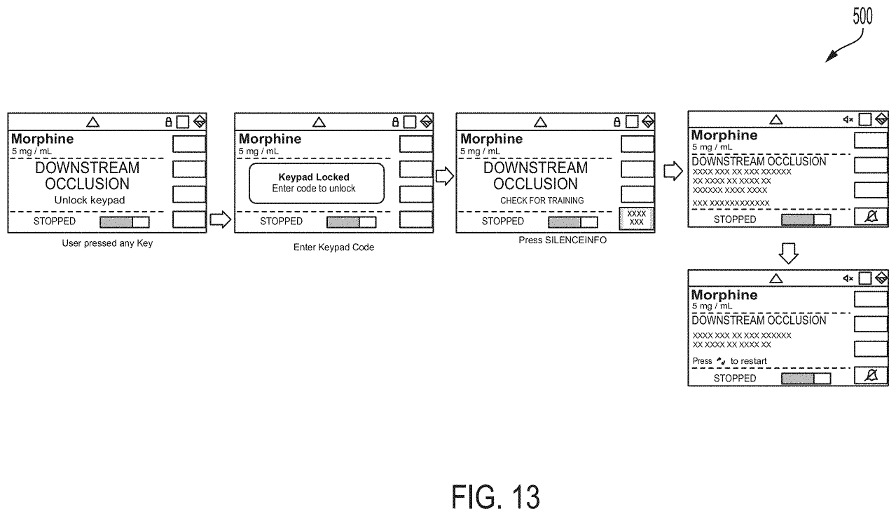

FIG. 13 illustrates a flow diagram of a downstream occlusion alarm 500 sequence of the infusion pump security system 150, according to an example of the present disclosure. In an embodiment, the pump generates a high priority downstream occlusion alarm 500 when the occlusion pressure setting is reached. In an embodiment, downstream in-line pressure (at 1.0 meter) is within the upper and lower limits (inclusive) for pressure settings less than or equal to the following (under nominal conditions from 0.1 to 250 ml/hr). However, in additional embodiments, the system may be configured to trigger a downstream occlusion alarm sequence in response to other downstream in-line pressure readings.

In an embodiment, when normal occlusion detection is activated by the user, the pump stops the infusion and generates a high priority downstream occlusion alarm 500 when the downstream pressure approximately one meter from the pump has exceeded the threshold pressure (psi) within the time indicated for various syringe sizes. When rapid occlusion detection is activated by the user and either the set is occluded before an infusion has started or the set is occluded after infusion reaches steady state, the pump will stop infusion and generate a high priority downstream occlusion alarm when the downstream pressure approximately one meter from the pump has exceeded the threshold pressure (psi) within the time indicated for various syringe sizes, flow rates and alarm settings. In the event an unauthorized user attempts to tamper with the administration set by applying pressure to the syringe barrel to dispense medication, the resulting increased pressure within the syringe is detected and accordingly, the pump will generate the downstream occlusion alarm. Additionally, the flange holder creates a physical limitation to the amount of medication that can be dispensed in this manner. In an embodiment, any attempts to twist or pull the intravenous (“IV”) set may also be detected by the downstream occlusion alarm depending on the degree to which the IV set becomes occluded during these attempts.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. Also, it should be appreciated that the features of the dependent claims may be embodied in the systems, methods, and apparatus of each of the independent claims.

Many modifications to and other embodiments of the invention set forth herein will come to mind to one skilled in the art to which these inventions pertain, once having the benefit of the teachings in the foregoing descriptions and associated drawings. Therefore, it is understood that the inventions are not limited to the specific embodiments disclosed, and that modifications and other embodiments are intended to be included within the scope of the appended claims. Although specific terms are employed herein, they are used in a generic and descriptive sense only and not for purpose of limitation.

1. A patient-controlled analgesia pump comprising:
an actuator;
a syringe holder;
a display screen;
a security door assembly;
a security frame assembly;
a swivel door assembly;
a plurality of sensors, including at least one occlusion sensor and at least one downstream inline pressure sensor;
a syringe flange;
a syringe push button;
a syringe barrel clamp;
a syringe pump lever;
a processor; and
a memory storing instructions, which when executed by the processor, cause the processor to
generate a syringe mis-loaded alarm when a syringe is mis-loaded into the PCA pump;
generate a syringe load error alarm when there is a syringe load error in the PCA pump;
generate a door unlocked alarm when the security door assembly is unlocked from the security frame assembly; and
generate a downstream occlusion alarm when there is a downstream occlusion in the PCA pump,
wherein the alarm is an auditory, graphical, visual, and/or tactile notification, or a combination thereof, and
wherein user-inputted authentication commands allow the user to silence and/or stop the alarm or alarms.

2. The pump of claim 1, wherein the syringe mis-loaded alarm is triggered when the security door assembly is closed and confirmed locked by the user, the pump is in a delivery mode, and any of the following conditions are true, except when the syringe mis-loaded alarm is active: the syringe flange is removed; the syringe push button is not in contact with the occlusion sensor; the syringe barrel clamp is opened; the syringe pump lever is activated; and/or the syringe push button detects high pressure reading.

3. The pump of claim 2, wherein the syringe mis-loaded alarm is triggered and the pump stops the delivery mode, when the pump is in delivery mode and any of the following conditions are true: the syringe flange is removed; the syringe push button is not in contact with the occlusion sensor; the syringe barrel clamp is opened; and/or syringe pump lever is activated.

4. The pump of claim 1, wherein the syringe load error alarm is triggered when the security door assembly is closed and confirmed locked by the user, the pump is not in a delivery mode, or is in a stopped state, the syringe push button detects a high pressure reading, and a syringe load point gets actuated.

5. The pump of claim 1, wherein the door unlocked alarm is triggered when the security door assembly is opened and the pump is in delivery mode.

6. The pump of claim 1, wherein the downstream occlusion alarm is triggered when the downstream in-line sensor detects pressure outside a user-inputted pressure range at about 1.0 meter downstream.

7. The pump of claim 1, wherein the security door assembly further comprises mating portions and the security frame assembly further comprises mating portions.

8. The pump of claim 7, wherein the mating portions on the security door assembly selectively engage the mating portions on the security frame assembly to selectively couple the security door assembly and the security frame assembly.

9. The pump of claim 1, wherein the security door assembly further comprises ribs, grooves and/or contours on the internal surface of the security door assembly.

10. The pump of claim 1, wherein the security door assembly further comprises a receiver and the security frame assembly further comprises a lock assembly.

11. The pump of claim 10, wherein the lock assembly selectively mates with the receiver.

12. A patient-controlled analgesia pump comprising:
an actuator;
a syringe holder;
a display screen;
a security door assembly, including a receiver;
a security frame assembly, including a lock assembly;
a swivel door assembly, including a syringe pump lever in an upright and depressed orientation; and
a plurality of sensors including at least one occlusion sensor and at least one downstream inline pressure sensor,
wherein the security door assembly interlocks with the security frame assembly and the lock assembly mates with the receiver.

13. The pump of claim 12, wherein the swivel door assembly further comprises a main assembly body to which the lever door pivotably couples, a swivel door that is pivotably attached to the assembly body and that includes side walls, a side wall plate coupled to the main assembly body so as to fully cover the syringe pump lever, a lever stopper, and a pull knob stopper.

14. The pump of claim 13, wherein the swivel door has an internal curvature that interlocks with the plunge lever and redirects force applied on the syringe pump lever, such that the plunge lever cannot be depressed when the swivel door is in a closed configuration relative to the main assembly body.

15. The pump of claim 12, wherein the lever stopper is pivotably attached to the main assembly body and interlocks with the syringe pump lever to retain it in an upright orientation, and wherein the pull knob stopper includes a locking pin that mates with the lever stopper at a locking receptacle to selectively lock it when it interlocks with the syringe pump lever.

16. The pump of claim 15, wherein the pull knob lever includes a mechanism to orient the locking pin to engage with the locking receptacle, and wherein the user can selectively inactivate the mechanism, so as to selectively disengage the locking pin and locking receptacle.

17. The pump of claim 12, wherein when the security door assembly and security frame assembly interlock and are in a closed configuration, the syringe holder is inaccessible.

18. The pump of claim 12, wherein when the security door assembly and security frame assembly interlock and are in a closed configuration, the display screen is accessible.

19. The pump of claim 12, wherein when the security door assembly and the security frame assembly interlock and are in a closed configuration, the swivel door assembly is inaccessible.

20. The pump of claim 12, wherein when the security door assembly and security frame assembly interlock and are in a closed configuration, the actuator is inaccessible.
